(19) United States
(12) Patent Application Publication
(10) Pub. No.: US 2025/0230114 A1
(43) Pub. Date: Jul. 17, 2025

(54) PROCESS FOR PRODUCING VINYL CHLORIDE MONOMER FROM ACETYLENE

(71) Applicant: JOHNSON MATTHEY PUBLIC LIMITED COMPANY, London (GB)

(72) Inventors: Martin Lucas SMIDT, London (GB); Joost SMIT, London (GB)

(21) Appl. No.: 18/852,883

(22) PCT Filed: May 30, 2023
(86) PCT No.: PCT/GB2023/051414

§ 371 Date: Sep. 30, 2024

(30) Foreign Application Priority Data
Jun. 10, 2022 (GB) 2208493.3

(51) Int. Cl. C07C 17/08; B01D 53/00

(52) U.S. Cl. CPC C07C 17/08; B01D 53/002

(57) ABSTRACT

The specification describes a process for the production of vinyl chloride monomer (VCM) from acetylene and HCl. The process involves a primary reactor and a secondary reactor each comprising a hydrochlorination catalyst. The feed from the primary reactor is sent to a knockout drum. The feed from the secondary reactor is split, part is combined with the feed from the primary reactor while the other part is sent to a vent recovery unit. The vent recovery unit includes one or more stages in which liquid containing VCM is separated and sent directly to the KO drum. A cold vapor is generated from the KO drum which is sent to a compressor, followed by a lights separation unit and a lights column.

### FIELD OF THE INVENTION

The present invention relates to a process for the conversion of acetylene to vinyl chloride monomer (VCM).

### BACKGROUND

The hydrochlorination of acetylene to produce VCM as the precursor to polyvinyl chloride (PVC) is currently a large scale industrial process, particularly in coal rich areas such as China and in areas rich in natural gas through natural gas to acetylene routes. Over 20 million tonnes of VCM are produced annually through acetylene hydrochlorination with the vast majority utilising mercuric chloride (HgCl₂) catalysts supported on activated carbon.

U.S. Pat. No. 3,268,299 (Crawford & Russell Inc) describes an apparatus for effecting catalytic reactions in a fixed bed reactor and can be used for reactions including the reaction between acetylene and hydrogen chloride to produce vinyl chloride. The apparatus reduces hot spot formation during the highly exothermic hydrochlorination reaction. FIG. 8 illustrates a typical setup process.

CN1884241A (Haiji) describes a process in which acetylene and hydrogen chloride are reacted together in a first stage reactor at a temperature of 100-180° C. in the presence of a HgCl₂/C catalyst to produce a crude vinyl chloride mixture. The crude mixture is cooled to around 40° C., compressed, cooled, condensed to remove VCM, and the uncondensed gas are compressed and reacted together in a second stage reactor at a temperature of 100-180° C. and pressure of 270 kPa in the presence of a HgCl₂/C. Incondensables from the second stage reactor are combined with the product from the first stage reactor before cooling.

A problem with this arrangement is that the compressor and the compressor outlet cooler are prone to fouling by heavies which are produced when VCM is exposed to temperatures above 100° C. The heat exchanged (Q) in the outlet cooler is proportional to the area (A), temperature difference (ΔT) and the heat transfer coefficient (U), i.e. Q=U·A·ΔT. As the surface becomes fouled, U is reduced and so is Q. For this reason, the cooler has to be sized with a sufficiently high area to accommodate for the fouling, which makes the cooler more expensive. If fouling becomes too severe then the process needs to be shut down to allow the fouling to be removed, which is expensive.

It would be advantageous if the process could be adapted to minimise fouling in the compressor and compressor outlet cooler, thereby avoiding downtime and avoiding the requirement to oversize the compressor cooler. The present invention addresses this problem.

### SUMMARY OF INVENTION

In the present invention a knockout (KO) drum is introduced before the compressor. The cooled product from the primary reactor is fed to the KO drum. A vapor (containing acetylene, HCl and residual VCM) is generated in the KO drum by evaporating liquid VCM in the KO drum which cools the contents of the drum. The KO drum is also fed with VCM-rich liquid separated from a vent recovery unit downstream from the secondary reactor. The vent recovery unit is operated in one or more stages and liquid from each stage is sent directly to the KO drum. The arrangement of the vent recovery and KO drum mean that the feed to the compressor can be cooled to a lower temperature than conventional processes, without requiring uneconomical amounts of cooling. The vapor has a low temperature, typically −10° C., which is much lower than the temperature of the feed to the compressor in CN1884241A (10-40° C.) and offers the benefits that: the compressor has to deal with a smaller volumetric throughput meaning that it can be smaller; and the temperature at the compressor outlet is lower, meaning that less fouling products are produced and the compressor and compressor outlet cooler are less prone to fouling.

The invention relates to a process for the production of vinyl chloride monomer (VCM), comprising the steps of:
feeding a feed stream comprising acetylene and HCl to a primary reactor and carrying out hydrochlorination in said primary reactor in the presence of a first hydrochlorination catalyst to produce a primary reactor product stream comprising vinyl chloride monomer along with unreacted acetylene and HCl;
cooling the primary reactor product stream and feeding the resulting cooled primary reactor product stream to a knockout (KO) drum;
generating vapor having a temperature of −20° C. to +10° C. by evaporating VCM in the KO drum;
feeding vapor from the KO drum to a compressor to produce a compressed stream;
feeding the compressed stream to a lights separation unit and separating the compressed stream into a liquid fraction and an overhead fraction containing unreacted acetylene, HCl and residual VCM;
sending the liquid fraction from the lights separation unit to a lights column and separating the liquid fraction into a bottom fraction containing VCM and an overhead fraction which is returned to the lights separation unit;
sending an overhead fraction from the lights separation unit to a secondary reactor and carrying out hydrochlorination in said secondary reactor in the presence of a second hydrochlorination catalyst to produce a secondary reactor product stream comprising vinyl chloride monomer along with unreacted acetylene and HCl;
splitting the secondary reactor product stream into a first portion which is sent to a vent recovery unit and a second portion which is combined with the primary reactor product stream;
condensing the first portion into a liquid in the vent recovery unit in one or more stages, and sending liquid from each stage directly to the KO drum.

### DESCRIPTION OF THE FIGURES

FIG. 1 illustrates a prior art process as described in CN1884241A.

FIG. 2 illustrates the process according to the present invention. A feed of acetylene and HCl (not shown) is fed to a primary reactor (21). The primary reactor product stream is cooled in a cooler (22) and fed to a knockout drum (23). A vapor is generated in the KO drum which is fed to a compressor (24). The compressed stream is fed to a lights separation unit (25). Liquid product separated in the lights separation unit is fed to a lights column (26) where it is separated into a stream containing VCM and heavies (27) and incondensables (28) which are returned to the lights separation unit. Incondensables (29) from the lights separation unit are fed to a secondary reactor (210). The product from the secondary reactor is cooled in a cooler (211) and split. A first portion (212a) is sent to a vent recovery unit (213) while a second portion (212b) is combined with the cooled product from the primary reactor upstream from the KO drum. Liquid VCM (214) separated in vent recovery is sent to the knockout drum. The vent recovery also produces a stream of inerts (215).

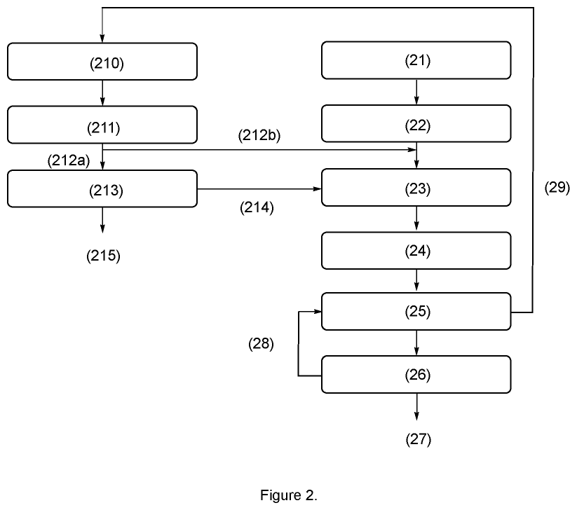

FIG. 3 illustrates a preferred embodiment of the lights separation unit (35). A compressed stream is fed sequentially to a first cooling stage (35a), a second cooling stage (35b) and a third cooling stage (35c). VCM is separated at each stage and collected in a receiver drum (316). Incondensables (39) from the third cooling stage are sent to a secondary reactor (not shown). Liquid VCM from is sent from the receiver drum to a lights column (36) where it is separated into a stream containing VCM and heavies (37) and incondensables (38) which are returned to the lights separation unit. As shown the incondensables (38) are combined with the stream entering the third cooling stage (35c) but the incondensables can be returned to the lights separation unit at any appropriate point.

FIG. 4 illustrates a preferred embodiment of the vent recovery. Cooled product from the secondary reactor is sent to a first cooling stage (413a) where it is separated into a liquid VCM stream (414a) which is sent to the knockout drum (43), and an incondensables stream which is fed to a second cooling stage (413b). The second cooling stage produces a liquid VCM stream (414b) which is sent to the knockout drum, and an incondensables stream which is fed to a third cooling stage (414c). The third cooling stage produces a liquid VCM stream (414c) which is sent to the knockout drum, and a stream of inerts (415).

### DETAILED DESCRIPTION OF THE INVENTION

Where a temperature is reported as “approximately [ ] ° C.” the value may vary by ±5° C. For example, a temperature of “approximately −10° C.” should be understood as meaning a temperature anywhere from −15° C. to −5° C.

### Primary Reactor

The primary reactor includes a first hydrochlorination catalyst which is active for the conversion of acetylene to VCM. Any suitable hydrochlorination catalyst may be used. In preferred embodiments the hydrochlorination catalyst contains gold. Gold catalysts have been well studied as hydrochlorination catalysts. Preferred catalysts include those described in WO2013/008004 and WO2020/254817 (Johnson Matthey) the contents of which are incorporated herein by reference. A particularly preferred catalyst comprises a complex of gold with a thiosulphate ligand on a carbon support.

In a preferred embodiment the primary reactor is a shell-and-tube reactor.

The feed to the primary reactor is a mixture of acetylene and HCl, typically at a molar ratio of approximately 50:50. There is preferably a slight excess of HCl to ensure that the catalyst remains in the active state. The feed to the primary reactor typically has a pressure of 0 to 4 barg (i.e. 1 to 5 bara), preferably 0 to 1 barg (i.e. 1 to 2 bara), such as 0 to 0.8 barg or 0.2 to 0.6 barg. Higher pressures, e.g. as high as 20 barg, are also possible.

Acetylene hydrochlorination is exothermic and the product stream from the primary reactor is a hot gas containing a mixture of VCM, acetylene and HCl. The primary reactor product stream is cooled before being sent to the KO drum, preferably using cooling water. Cooling at this stage reduces the duty on the KO drum. Typically cooling water is supplied at 30° C. and the secondary reactor product stream is cooled to 40° C. The cooled stream is referred to as the cooled primary reactor product stream. The cooled primary reactor stream is preferably passed through a filter before being fed to the KO drum.

As will be described in more detail under the “secondary reactor” heading below, the product stream from the secondary reactor is split into a first portion which is sent to the vent recovery unit and a second portion which is combined with the product stream from the primary reactor. It is preferred that the secondary reactor product stream is cooled before being split. This is preferred because the product stream from the secondary reactor is at a higher pressure than the product stream from the primary reactor, and cooling of the secondary reactor product stream can be achieved in a smaller cooling unit which is more efficient. It is therefore preferred that the secondary reactor product stream is cooled to produce a cooled secondary reactor product stream, then split into a first portion which is sent to vent recovery and a second portion which is combined with the cooled primary reactor product stream. While it is possible to split the secondary reactor product stream before any cooling and combine it with the primary reactor product stream, this is a less preferred option.

### KO Drum

The role of the KO drum is to generate a cold vapor, typically at a temperature of approximately −10° C., which is fed to the compressor. The KO drum receives the cooled primary reactor stream and liquid fractions from the vent recovery, described in detail under the “vent recovery” heading. The vapor is created by evaporating the liquid fractions from vent recovery, containing VCM as the major component, which cools the entire contents of the KO drum. Typically the liquid fractions from vent recovery are sprayed into the KO drum to create a high surface area for evaporation. Additional liquid VCM, e.g. a portion of the reflux from the lights separation unit or lights column, can be sent to the KO drum in order to achieve additional cooling, e.g. if the amount of VCM from vent recovery is insufficient to cool the vapor to a suitable temperature.

### Compressor

The secondary reactor operates at a higher pressure than the primary reactor, meaning that the primary reactor product stream has to be compressed. A compressor is located downstream from the KO drum which increases the pressure of the vapor before the lights separation unit and the lights column. Any suitable compressor may be used, but a preferred type is a screw compressor because these are generally less sensitive to fouling and cheaper than centrifugal compressors. Oil-free screw compressors are most preferred.

The pressure at the outlet of the compressor will depend on the desired pressure in the secondary reactor. While pressures in the secondary reactor as high as 20 barg are possible, more typically the pressure in the secondary reactor is 2 to 5 barg, such as 3 to 5 barg. Accordingly, the vapor is compressed to 2 to 5 barg, such as 3 to 5 barg. To allow for pressure loss between the compressor and the secondary reactor (e.g. via the lights separation unit and lights column), the compressor typically compresses the vapor to a pressure about 0.5 bar above the pressure of the lights column. For instance, if the lights column operates at 3.5 barg then the compressor outlet should be 4 barg.

In the present invention vapor entering the compressor has a temperature of approximately −10° C. while the vapor exiting the compressor has a temperature of approximately 90° C. This contrasts with the situation in CN1884241A, where the temperature of the crude vinyl chloride stream fed to the compressor is 40° C. and is compressed to 370-400 kPa (g). Assuming a similar temperature increase across the compressor, the temperature of the compressed vapor would be expected to be around 140° C., and certainly above 100° C. which is a temperature at which fouling starts to occur. The lower exit temperature from the compressor in the instant process avoids fouling in the compressor and compressor outlet cooler.

It is preferred that the compressed stream is cooled before being sent to the lights separation unit. The purpose of this cooling is to lower the temperature of the feed to the lights separation unit, but without separating out a liquid stream. Water is particularly suitable for such cooling.

### Lights Separation Unit

The role of the lights separation unit is to separate the compressed stream into a liquid fraction containing VCM as the major product, and an overhead fraction containing unreacted acetylene, HCl and residual VCM, which is sent to the secondary reactor.

The liquid fraction, or fractions, produced by the lights separation unit are preferably collected in a receiver drum before being sent to the lights column. Some of the liquid from the receiver drum may also be sent to the KO drum in order to maintain of the vapor from the KO drum at a sufficiently low temperature. Any incondensables from the receiver drum may be sent to the secondary reactor.

The lights separation unit comprises at least one cooling stage in which a liquid fraction is separated. It is preferred that the lights separation unit comprises a series of cooling stages carried out at decreasing temperatures (i.e. using coolants of decreasing temperatures) with a liquid fraction being separated at each stage and the overhead fraction being sent to the next cooling stage. As noted above, the liquid fractions are preferably collected in a receiver drum before being sent to the lights column. The fractions may be combined or each sent separately to the receiver drum. The overhead from the final cooling stage is fed to the secondary reactor.

In a particularly preferred embodiment the lights separation unit comprises: a first cooling stage using a coolant at a temperature of approximately +3° C.; a second cooling stage using a coolant at a temperature of approximately −10° C.; a third cooling stage using a coolant at a temperature of approximately −25° C. In each case, the liquid fractions obtained on the process side are approximately 5° C. above the temperature of the respective coolant. The overhead from the first cooling stage is fed to the second cooling stage; the overhead from the second cooling stage is fed to the third cooling stage; and the overhead from the third cooling stage is fed to the secondary reactor.

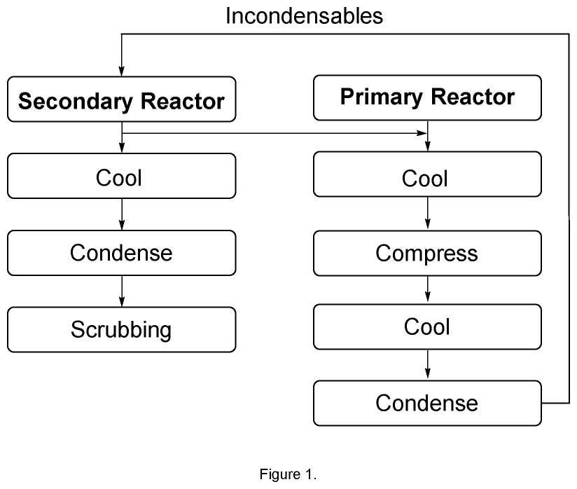

To the inventors' best knowledge, while previously described VCM plants may cool the compressed gas in a series of stages, separating out a liquid fraction at each stage has not been described. As is shown in FIG. 1 of CN1884241A, there is only a single condensing unit meaning that all of the compressed stream is cooled to the lowest temperature of the refrigeration unit. The result was that there was a large duty on the condenser and VCM distillation column which has to reboil a cold liquid. Separating a liquid fraction at each stage is therefore more efficient and reduces the duty on the lights separation unit and the lights column.

### Lights Column

The role of the lights column is to separate the liquid fractions from the lights separation unit into a bottom fraction containing VCM and an overhead fraction. This can be achieved by conventional distillation using a multistage distillation column. The overhead fraction from the lights column is returned to the lights separation unit. The bottom fraction contains VCM and heavies and is preferably sent to further purification to remove heavies. Some of the bottom fraction may also be sent to the KO drum as described under the “KO drum” heading, but it is preferable that the liquid from the receiver drum is used for this purpose.

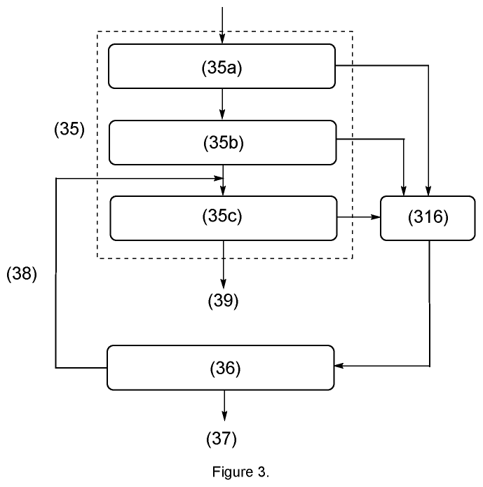

A flow diagram of a preferred lights separation unit and lights column is shown in FIG. 3. A compressed stream from the compressor, which has normally been cooled e.g. using water (not shown), enters a first cooling stage within the lights separation unit. A liquid fraction from the first cooling stage is collected in a drum and the incondensable fraction is sent to a second cooling stage, operating at a lower temperature than the first cooling stage. The process is repeated with the incondensables from the second cooling stage being sent to a third cooling stage operating at a lower temperature. The incondensables from the third cooling stage are send to the secondary reactor. The combined liquid fractions are collected in a receiver drum and then sent to the lights column where they are separated into a bottom fraction which is sent for heavies separation. The incondensables are returned to the lights separation unit.

### Secondary Reactor

Overheads from the lights separation unit are sent to the secondary reactor. Conversion in the primary reactor is typically good and conversions in excess of 85% are achievable, for instance when using a gold thiosulphate complex catalyst as described in WO2013/008004 and available from Johnson Matthey under the brand PRICAT™ MFC. The throughput to the secondary reactor is therefore much less than the primary reactor and the secondary reactor capacity can be much smaller than the primary reactor. The secondary reactor needs to be designed to handle the higher pressure feed. The pressure of the feed may be as high as 20 barg, but typically is at a pressure of 2-3 barg.

The secondary reactor includes a second hydrochlorination catalyst. Any suitable hydrochlorination catalyst may be used In preferred embodiments the hydrochlorination catalyst contains gold. Gold catalysts have been well studied as hydrochlorination catalysts. Preferred catalysts include those described in WO2013/008004 and WO2020/254817 (Johnson Matthey) the contents of which are incorporated herein by reference. For the avoidance of doubt, the second hydrochlorination catalyst may be the same as or different from the first hydrochlorination catalyst. A particularly preferred catalyst comprises a complex of gold with a thiosulphate ligand on a carbon support.

In a preferred embodiment the secondary reactor is a shell-and-tube reactor.

As was explained under the “primary reactor” heading, the secondary reactor product stream is split. A first portion is sent to the vent recovery unit and a second portion is combined with the primary reactor product stream. The relative proportion sent to the vent recovery unit will depend on the amount of amount of inerts in the system; the higher the amount of inerts the greater the proportion of secondary reactor product sent to the vent recovery unit.

It is preferred that the secondary reactor product stream is cooled before being split, preferably using cooling water. Typically cooling water is supplied at 30° C. and the secondary reactor product stream is cooled to 40° C. The cooled stream is referred to as the cooled secondary reactor product stream. The secondary reactor stream is preferably passed through a filter either before or after cooling.

### Vent Recovery Unit

The overhead from the lights column is returned to the lights separation unit. This means that any inerts (e.g. N₂, Ar etc. . . . ) which are present in the feed to the primary reactor could build up in the system. To avoid this, a vent recovery unit is included downstream from the secondary reactor. The role of the vent recovery unit is to separate inerts from the first portion from the secondary reactor product stream. The vent recovery unit also plays an important role in generating liquid VCM which is fed to the KO drum.

The vent recovery unit condenses the first portion into a liquid in one or more cooling stages. Liquid is generated at each stage, predominantly containing VCM and unreacted acetylene and HCl. Typically the liquid comprises about 90 mol % VCM with the remainder being acetylene and HCl. The liquid from each stage is sent directly to the KO drum. As used herein “directly” means that there is no combination of liquid from each stage until the KO drum. This is important because evaporating VCM in the KO drum is used to keep the temperature of the vapor to the compressor low. If the VCM fractions from the cooling stages were combined they could evaporate prematurely before the KO drum which would be wasteful because it would not contribute to cooling the product from the primary and secondary reactors.

In a preferred embodiment the vent recovery unit comprises a series of refrigeration stages operating at decreasing temperatures. In a particularly preferred embodiment the vent recovery unit comprises: a first cooling stage using a coolant at approximately −10° C. and a second cooling stage using a coolant at approximately −25° C. In a further preferred embodiment the vent recovery unit includes a third cooling stage using a coolant at approximately −65° C. In each case, the liquid fractions obtained on the process side are approximately 5° C. above the temperature of the respective coolant.

Suitable coolants will be known to those skilled in the art. Ethane is particularly suitable for generating a −65° C. coolant. Propane is particularly suitable for generating coolants at −25° C., −10° C. and +3° C.

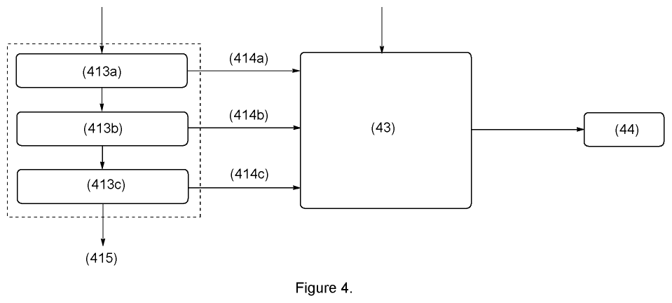

A flow diagram of a preferred vent recovery unit is shown in FIG. 4. The cooled secondary reactor product enters a first cooling stage within the vent recovery unit. A liquid fraction from the first cooling stage is sent to the KO drum while the incondensable fraction is sent to a second cooling stage, operating at a lower temperature than the first cooling stage. The process is repeated with the liquid fraction from the second cooling stage being sent to the KO drum and the incondensables from the second cooling stage being sent to a third cooling stage operating at a lower temperature. The incondensables from the third cooling stage (mainly inerts) are vented to avoid build-up in the system. The VCM evaporates in the KO drum, cooling the product from the primary and secondary reactors which is also fed to the KO drum. This creates a cold vapor which is sent to the compressor.

1. A process for the production of vinyl chloride monomer (VCM), comprising the steps of:
feeding a feed stream comprising acetylene and HCl to a primary reactor and carrying out hydrochlorination in said primary reactor in the presence of a first hydrochlorination catalyst to produce a primary reactor product stream comprising vinyl chloride monomer along with unreacted acetylene and HCl;
cooling the primary reactor product stream and feeding the resulting cooled primary reactor product stream to a knockout (KO) drum;
generating vapor having a temperature of −20° C. to +10° C. by evaporating VCM in the KO drum;
feeding vapor from the KO drum to a compressor to produce a compressed stream;
feeding the compressed stream to a lights separation unit and separating the compressed stream into a liquid fraction and an overhead fraction containing unreacted acetylene, HCl and residual VCM;
sending the liquid fraction from the lights separation unit to a lights column and separating the liquid fraction into a bottom fraction containing VCM and an overhead fraction which is returned to the lights separation unit;
sending an overhead fraction from the lights separation unit to a secondary reactor and carrying out hydrochlorination in said secondary reactor in the presence of a second hydrochlorination catalyst to produce a secondary reactor product stream comprising vinyl chloride monomer along with unreacted acetylene and HCl;
splitting the secondary reactor product stream into a first portion which is sent to a vent recovery unit and a second portion which is combined with the primary reactor product stream;
condensing the first portion into a liquid in the vent recovery unit in one or more stages, and sending liquid from each stage directly to the KO drum.

2. A process according to claim 1, wherein the primary reactor product stream is cooled using cooling water.

3. A process according to claim 1, wherein the vapor is compressed to a pressure of 2 to 5 barg.

4. A process according to claim 1, wherein the compressed stream is cooled before being sent to the lights separation unit.

5. A process according to claim 1, wherein the compressed stream is cooled using water before being sent to the lights separation unit.

6. A process according to claim 1, wherein the lights separation unit comprises a series of cooling stages carried out at decreasing temperatures with a liquid fraction being separated at each stage and the overheads being sent to the next cooling stage.

7. A process according to claim 6, wherein the lights separation unit comprises:
a first cooling stage using a coolant at a temperature of approximately +3° C.;
a second cooling stage using a coolant at a temperature of approximately −10° C.;
a third cooling stage using a coolant at a temperature of approximately −25° C.

8. A process according to claim 1, wherein the vent recovery unit comprises a series of cooling stages carried out at decreasing temperatures.

9. A process according to claim 8, wherein the vent recovery unit comprises:
a first cooling stage using a coolant at a temperature of approximately −10° C.;
a second cooling stage using a coolant at a temperature of approximately −25° C.;
a third cooling stage using a coolant at a temperature of approximately −65° C.

10. A process according to claim 1, wherein the liquid fraction(s) produced by the lights separation unit are collected in a receiver drum before being sent to the lights column.

11. A process according to claim 1, wherein the secondary reactor product stream is cooled before being split.

12. A process according to claim 11, wherein the secondary reactor product stream is cooled using cooling water to a temperature of approximately 40° C.

13. A process according to claim 1, wherein the first hydrochlorination catalyst and/or the second hydrochlorination catalyst is a catalyst comprising a complex of gold with a thiosulphate ligand on a carbon support.

14. A process according to claim 1, wherein the primary reactor and/or secondary reactor is a shell-and-tube reactor.
